(19) United States
(12) Patent Application Publication
(10) Pub. No.: US 2010/0249550 A1
(43) Pub. Date: Sep. 30, 2010

(54) Method And Apparatus For Optical Filtering Of A Broadband Emitter In A Medical Sensor

(75) Inventor: David Lovejoy, Thiensville, WI (US)

Correspondence Address: NELLCOR PURITAN BENNETT LLC;ATTN: IP LEGAL, 6135 Gunbarrel Avenue, Boulder, CO, 80301, US

(73) Assignee: Neilcor Puritan Bennett LLC, Boulder, CO (US)

(21) Appl. No.: 12/411,213

(22) Filed: Mar. 25, 2009

(51) Int. Cl. A61B 5/1455

(52) U.S. Cl. 600/323

(57) ABSTRACT

A system and method for determining physiological parameters of a patient based on light transmitted through the patient. The light may be transmitted via a broadband light source and received by a detector The light may also be optically filtered by an optical filter of either the light source or the detector. Based on the filter, specific wavelengths of light are received by the detector for use in monitoring the physiological parameters of the patient.

### BACKGROUND

The present disclosure relates generally to medical devices and, more particularly, to sensors used for sensing physiological parameters of a patient.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed or scattered. More specifically, the light passed through the tissue is typically selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

The light sources utilized in pulse oximeters typically must be selected based on their ability to transmit light at specific wavelengths so that the absorption and/or scattering of the transmitted light in a patient's tissue may be properly determined. This may preclude the use of a multitude of readily available, and typically less costly, light sources that transmit light at various wavelengths.

### BRIEF DESCRIPTION OF THE DRAWINGS

Advantages of the disclosure may become apparent upon reading the following detailed description and upon reference to the drawings in which:

FIG. 1 illustrates a perspective view of a pulse oximeter in accordance with an embodiment;

FIG. 2 illustrates a simplified block diagram of a pulse oximeter in FIG. 1, according to an embodiment;

FIG. 3 illustrates a simplified block diagram of a pulse oximeter in FIG. 1, according to a second embodiment;

FIG. 4 illustrates a simplified block diagram of a pulse oximeter in FIG. 1, according to a third embodiment; and

FIG. 5 illustrates a simplified block diagram of a pulse oximeter in FIG. 1, according to a fourth embodiment.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Sensors for pulse oximetry or other applications utilizing spectrophotometry are provided therein that include the use of broadband emitters that emit light at in a range of wavelengths. This transmitted light may be filtered by optical filters that may be located either adjacent the broadband emitter or adjacent the detector. In one embodiment, multiple detectors may be utilized for reception of light from a single emitter. The multiple detectors may each be able to generate signals based on the light received from the broadband emitter, and transmit the generated signals across independent channel lines associated with each of the multiple detectors. A monitor in the pulse oximeter system may receive the signals and calculate physiological parameters of a patent based on the signals without having to demodulate the received signals first.

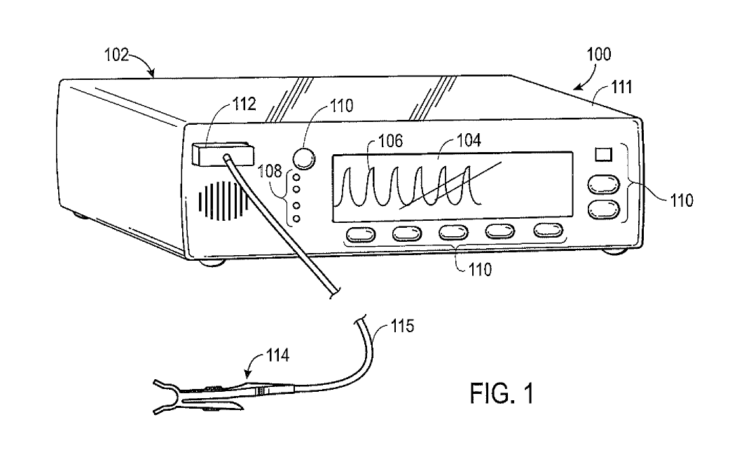

Turning to FIG. 1, a perspective view of a medical device is illustrated in accordance with an embodiment. The medical device may be a pulse oximeter 100. The pulse oximeter 100 may include a monitor 102, such as those available from Nellcor Puritan Bennett LLC. The monitor 102 may be configured to display calculated parameters on a display 104. As illustrated in FIG. 1, the display 104 may be integrated into the monitor 102. However, the monitor 102 may be configured to provide data via a port to a display (not shown) that is not integrated with the monitor 102. The display 104 may be configured to display computed physiological data including, for example, an oxygen saturation percentage, a pulse rate, and/or a plethysmographic waveform 106. As is known in the art, the oxygen saturation percentage may be a functional arterial hemoglobin oxygen saturation measurement in units of percentage SpO₂, while the pulse rate may indicate a patient's pulse rate in beats per minute. The monitor 102 may also display information related to alarms, monitor settings, and/or signal quality via indicator lights 108.

To facilitate user input, the monitor 102 may include a plurality of control inputs 110. The control inputs 110 may include fixed function keys, programmable function keys, and soft keys. Specifically, the control inputs 110 may correspond to soft key icons in the display 104. Pressing control inputs 110 associated with, or adjacent to, an icon in the display may select a corresponding option. The monitor 102 may also include a casing 111. The casing 111 may aid in the protection of the internal elements of the monitor 102 from damage.

The monitor 102 may further include a sensor port 112. The sensor port 112 may allow for connection to an external sensor 114, via a cable 115 which connects to the sensor port 112. The sensor 114 may be of a disposable or a non-disposable type. Furthermore, the sensor 114 may obtain readings from a patient, which can be used by the monitor to calculate certain physiological characteristics such as the blood-oxygen saturation of hemoglobin in arterial blood, the volume of individual blood pulsations supplying the tissue, and/or the rate of blood pulsations corresponding to each heartbeat of a patient.

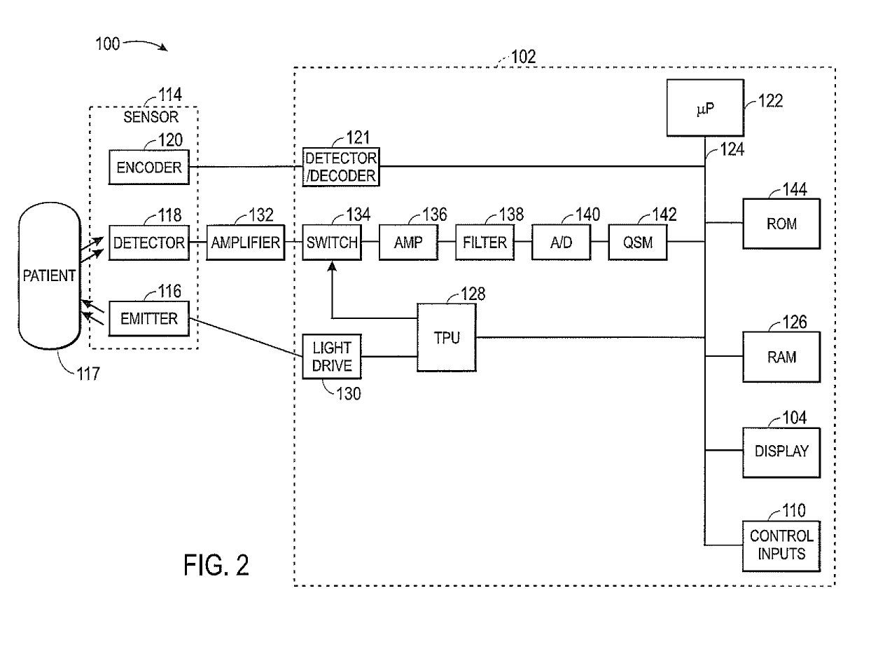

Turning to FIG. 2, a simplified block diagram of a pulse oximeter 100 is illustrated in accordance with an embodiment. Specifically, certain components of the sensor 114 and the monitor 102 are illustrated in FIG. 2. The sensor 114 may include an emitter 116, a detector 118, and an encoder 120. It should be noted that the emitter 116 may be capable of emitting at least two wavelengths of light, e.g., RED and infrared (IR) light, into the tissue of a patient 117 to calculate the patient's 117 physiological characteristics, where the RED wavelength may be between about 600 nanometers (nm) and about 700 nm, and the IR wavelength may be between about 800 nm and about 1000 nm. A single broadband light source may be used as the emitter 116, whereby the broadband light source may transmitting light at various wavelengths, including the RED and IR wavelengths, for use in measuring, for example, water fractions, hematocrit, or other physiologic parameters of the patient 117. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In one embodiment, the detector 118 may be capable of detecting light at various intensities and wavelengths. In operation, light enters the detector 118 after passing through the tissue of the patient 117. The detector 118 may convert the light at a given intensity, which may be directly related to the absorbance and/or reflectance of light in the tissue of the patient 117, into an electrical signal. That is, when more light at a certain wavelength is absorbed or reflected, less light of that wavelength is typically received from the tissue by the detector 118. After converting the received light to an electrical signal, the detector 118 may send the signal to the monitor 102, where physiological characteristics may be calculated based at least in part on the absorption of light in the tissue of the patient 117.

Additionally the sensor 114 may include an encoder 120, which may contain information about the sensor 114, such as what type of sensor it is (e.g., whether the sensor is intended for placement on a forehead or digit) and the wavelengths of light emitted by the emitter 116. This information may allow the monitor 102 to select appropriate algorithms and/or calibration coefficients for calculating the patient's physiological characteristics. The encoder 120 may, for instance, be a memory on which one or more of the following information may be stored for communication to the monitor 102: the type of the sensor 114; the wavelengths of light emitted by the emitter 116; and the proper calibration coefficients and/or algorithms to be used for calculating the patient's 117 physiological characteristics.

In another embodiment, the encoder 120 may be removed from the sensor 114. For example, if a broadband emitter 116 is utilized with an optical filter that allows only light of a certain wavelength to pass to the detector 118, then there may be no need for the transmission of information related to wavelengths of light emitted by the emitter 116 and the proper calibration coefficients and/or algorithms to be used for calculating the patient's 117 physiological characteristics Instead, the actual wavelengths of light received will correspond to the wavelengths passed by the optical filter, and no calibration coefficients and/or algorithms will be utilized to calculate the patient's 117 physiological characteristics. Accordingly, the encoder 120 may be removed from the sensor 114.

Signals from the detector 118 and the encoder 116 (if utilized) may be transmitted to the monitor 102. The monitor 102 may include one or more processors 122 coupled to an internal bus 124. Also connected to the bus may be a RAM memory 126 and a display 104. A time processing unit (TPU) 128 may provide timing control signals to light drive circuitry 130, which controls when the emitter 116 is activated, and if multiple light sources are used, the multiplexed timing for the different light sources. TPU 128 may also control the gating-in of signals from detector 118 through an amplifier 132 and a switching circuit 134. These signals are sampled at the proper time, depending at least in part upon which of multiple light sources is activated, if multiple light sources are used. The received signal from the detector 118 may be passed through an amplifier 136, a low pass filter 138, and an analog-to-digital converter 140 for amplifying, filtering, and digitizing the electrical signals the from the sensor 114. The digital data may then be stored in a queued serial module (QSM) 142, for later downloading to RAM 126 as QSM 142 fills up. In an embodiment, there may be multiple parallel paths of separate amplifier, filter, and A/D converters for multiple light wavelengths or spectra received.

In an embodiment, based at least in part upon the received signals corresponding to the light received by detector 118, processor 122 may calculate the oxygen saturation using various algorithms. These algorithms may require coefficients, which may be empirically determined, and may correspond to the wavelengths of light used. The algorithms may be stored in a ROM 144 and accessed and operated according to processor 122 instructions.

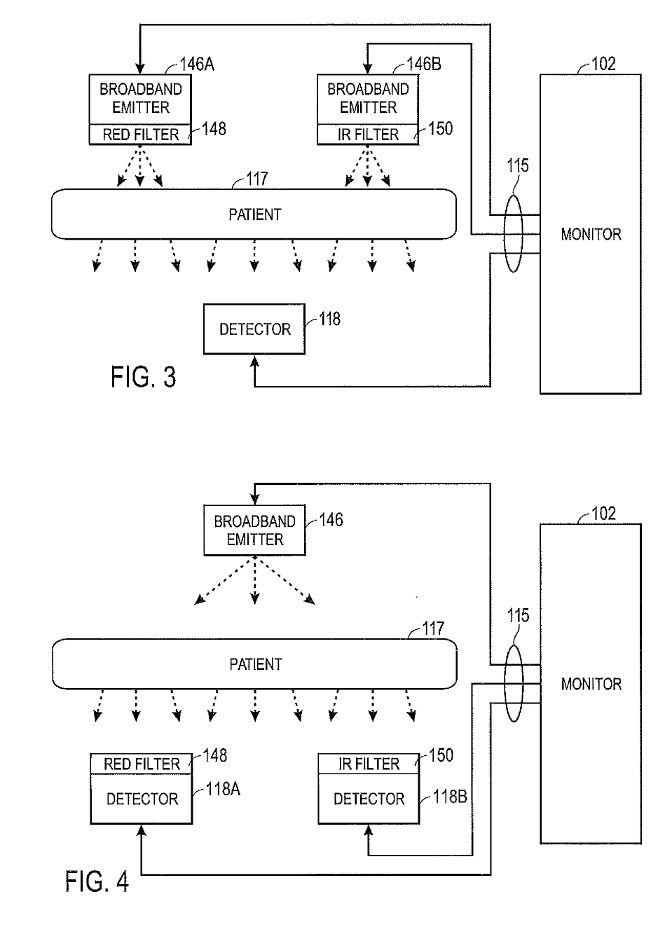

FIG. 3 illustrates an embodiment that may include two broadband emitters 146A and 146B and one detector 118 in sensor 114 Unlike a typical sensor that may include a first emitter that may transmit light in a visible frequency, such as 660 nm as well as a second emitter that may transmit light in an infrared (IR) range such as approximately 900 nm, the sensor assembly 114 of FIG. 3 may include two broadband emitters 146A and 146B that may transmit light across multiple wavelengths. For example, the broadband emitters 146A and 146B may be light emitting diodes (LEDs) that transmit light at wavelengths between, for example, 380 nm and 2500 nm. As such, the broadband emitters 146A and 146B may transmit light of wavelengths for across both visible and infrared wavelengths. Accordingly, processes such as binning, which may be defined as the process of selecting LEDs that may transmit at specific frequencies, such as 660 nm and 900 nm, may be avoided. Because the LEDs do not have to be binned to perform at a certain wavelength, more LEDs may be available for use in the system illustrated in FIG. 3. That is, broadband emitters, such as LEDs, are no longer excluded from use because of an inability to transmit light at a peak wavelength ranges used by the monitor 102.

Instead, a visible light optical filter 148 that may, for example, allow only a single wavelength or a range of red light (between the total range of red light from about 600-700 nm) to pass through the optical filter 148, may be used with one of the broadband emitters, for example, 146A. Similarly, an infrared (IR) filter 150 that may, for example, allow only a single wavelength or a range of IR light (between a range of IR light from about 700 nm to 1400 nm), may be used with another broadband emitter, for example, 146B. Through use of the optical filters 148 and 150, the light from the broadband emitters 146A and 146B may be filtered so that only a single wavelength, or a specified range of light, for each emitter 146A and 146B is transmitted to the patient 117.

The optical filters 148 and 150 may, for example, be integrated into the die package of the respective broadband emitters 146A and 146B. For example, each optical filter 148 and 150 may be applied via, for example, thin film deposition over the emitters 146A and 146B. Alternatively, the optical filters 148 and 150 may be disposed adjacent the broadband emitters 146A and 146B, such that the filters 148 and 150 may be separate from the die packages of the broadband emitters 146A and 146B. In this embodiment, the optical filters 148 and 150 may be applied to glass, for example, to generate filter glass that may lie adjacent to the broadband emitters 146A and 146B. In this manner, the filter glass may be disposed between the broadband emitters 146A and 146B and the detector 118.

The broadband emitters 146A and 146B may receive input signals from monitor 102. These input signals may be used to activate the broadband emitters 146A and 146B so that light may be generated via the emitters 146A and 146B. For example, emitter 146A may be activated while emitter 146B receives no input signal, thus remaining deactivated. This period of activation of the emitter 146A may be followed by a period of no input signals being delivered to the emitters 146A and 146B, i.e. a dark interval. Subsequently, an activation signal may be transmitted to emitter 146B while emitter 146A receives no input signal, thus remaining deactivated. In this manner, the emitter 146A and the emitter 146B may be alternately activated to each generate light during an independent period of time.

As the light is generated from the respective emitters 146A and 146B, the light passes through the respective red filter 148 and IR filter 150 corresponding to each broadband emitter 146A and 146B. For example, the red filter 148 may allow visible light in the optical range of about 660 nm to pass into the patient. Additionally, for example, the IR filter 150, may allow light at approximately 900 nm to pass into the patient 117. Accordingly, the emitters 146A and 146B may alternately transmit filtered light through the patient 117 for detection by the detector 118.

This received light may be scattered and/or absorbed by the patient 117, and may subsequently exit the patient 117. Upon exiting the patient 117, the light may be detected by the detector 118. The detector 118 may detect the light, which may include both visible and IR wavelength light, and may generate electrical signals corresponding to the detected light. To aid in the interpretation of these signals, a demodulator may be utilized. The demodulator may interpret the various received signals as, for example, corresponding to light in either the red or infrared spectrum. This demodulation may, for example, take place in the monitor 102. That is, the received signals at detector 118 may be transmitted via cable 115 to the monitor 102 for processing, which may include demodulation of the signals transmitted from the detector 118. Based on these demodulated signals, the oxygenation of the blood of the patient 117 may be determined in accordance with known techniques.

While a pulse oximeter 100 utilizing a demodulator was described above with respect to FIG. 3, alternate configurations of the pulse oximeter 100 may be implemented without the use of a demodulator. FIG. 4 illustrates one such configuration of a pulse oximeter 100 that may operate without a demodulator. The pulse oximeter 100 of FIG. 4 may include a sensor 114 with a single broadband emitter 146 as well as two detectors 118A and 118B connected to the monitor 102 via a cable 115. The broadband emitter 146 may transmit light across a given range of wavelengths that may include, for example, both visible and IR light. This light may pass into patient 117, and may pass from patient 117 to each of the detectors 118A and 118B through, for example, an optical filter 148 and 150. As discussed below, the optical filters 148 and 150 allow the detectors 118A and 118B to each receive separate wavelengths of light, and thus, generate separate signals corresponding to the received light. Accordingly, a demodulator is not required because the signals corresponding to, for example, visible and IR light, are already separated from each other via the independent detectors 118A and 118B.

Accordingly, the first detector 118A may be associated with an optical filter 148, which may allow light of a given wavelength, such as light in the red spectrum around 660 nm, or a given range of wavelengths to pass to the detector 118A. Similarly, the second detector 118B may be associated with to an optical filter 150, which may allow light of a given wavelength, such as light in the infrared spectrum around 900 nm, or a given range of wavelengths to pass to the detector 118B. The optical filters 148 and 150 may, for example, be integrated into the respective die package of the detectors 118A and 118B. Alternatively, the optical filters 148 and 150 may be positioned adjacent the detectors 118A and 118B, such that the filters 148 and 150 may be separate from the die packages of the detectors 118A and 118B as, for example, filter glass.

In operation, the pulse oximeter 100 of FIG. 4 may include a broadband emitter 146 that may receive electrical signals from the monitor 102 via the cable 115. These electrical signals may cause the broadband emitter 146 to transmit light in a given range of wavelengths, such as 380 nm to approximately 2500 nm. This light may be transmitted to the patient 117, and may pass through the patient 117 to the filters 148 and 150 of detectors 118A and 118B. The detector 118A, associated with the optical filter 148, may receive light in the visible light range, such as the red frequency range of light and may generate signals corresponding to the received light. These signals may be transmitted via an independent channel line, i.e. a signal path, to monitor 102 across cable 115. Similarly, the detector 118B, associated with the optical filter 150, may receive light in the infrared light range and may generate signals corresponding to the received light. These signals may be transmitted via a second independent channel line, i.e. a signal path, to monitor 102 across cable 115. Thus, the monitor 102 may receive two sets of signals indicative of light transmitted through the patient 117 across separate channels. As such, because the received signals may be on different channels, the signal transmitted from the detectors 118A and 118B to the monitor 102 may not need to be demodulated. Accordingly, this may reduce the cost and complexity of the monitor 102.

In an embodiment, detectors 118A and 118B may include UV enhanced silicon photodiodes. UV enhanced photodiodes may be designed for low noise detection in the UV region of electromagnetic spectrum. Inversion layer structure UV enhanced photodiodes may exhibit 100% internal quantum efficiency and may be well suited for low intensity light measurements They may have high shunt resistance, low noise and high breakdown voltages.

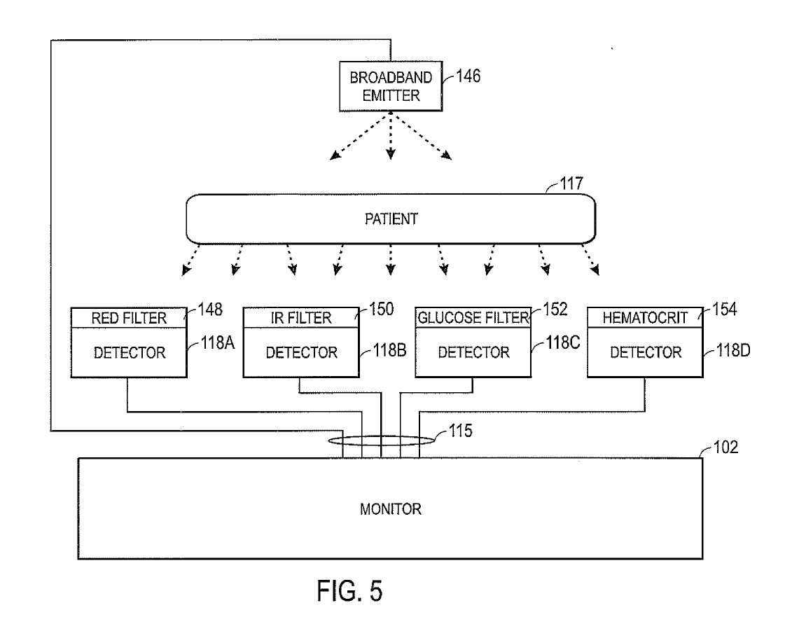

As discussed above with respect to FIG. 4, utilizing multiple detectors, such as detectors 118A and 118B, may be beneficial in that the multiple detectors may each utilize an independent signal path to transmit signals corresponding to received light, eliminating demodulation of the signals. Use of multiple detectors may also be beneficial when multiple physiological parameters of the patient 117 are to be monitored simultaneously FIG. 5 illustrates an embodiment whereby multiple physiological parameters of the patient 117 may be simultaneously monitored via a detector array.

FIG. 5 illustrates a pulse oximeter 100 that utilizes a detector array for simultaneous monitoring of multiple physiological parameters of a patient 117, as set forth above. The pulse oximeter 100 includes a single broadband emitter 146 with four detectors 118A, 118B, 118C, and 118D. The single broadband emitter 146 of FIG. 5 may operate in a substantially similar manner to the emitter 146 illustrated and described above with respect to FIG. 4. Furthermore, the detectors 118A-D may each be coupled to a respective optical filter 148, 150, 152, and 154. The first detector 118A may be associated with an optical filter 148, which may allow light of a given wavelength, such as light in the red spectrum around 660 nm, or a given range of wavelengths to pass to the detector 118A. Similarly, the second detector 118B may be associated with to an optical filter 150, which may allow light of a given wavelength, such as light in the infrared spectrum around 900 nm, or a given range of wavelengths to pass to the detector 118B. Additionally, a glucose filter 152, which may be associated with detector 118C, may allow light of a given wavelength, such as light at a wavelength of approximately 1000 nm, or a given range of wavelengths to pass to the detector 118C. Furthermore, a hematocrit optical filter 154, which may be associated with detector 118D, may allow light of a given wavelength, such as light at a wavelength of approximately 550 nm, or a given range of wavelengths to pass to the detector 118D.

In this manner, a single broadband emitter 146 may be utilized to transmit light to a plurality of detectors 118A-D, each with an optical filter 148, 150, 152, and 154 that specifically allows certain wavelengths of light to pass to the detectors 118A-D. By calibrating each of the filters 148, 150, 152, and 154 to pass a respective wavelength or range of wavelengths, the detectors 118A-D may each be able to receive light that may be utilized in detecting specific physiological parameters according to the light received.

Moreover, by utilizing multiple detectors 118, each with its own respective channel line to the monitor 102, the monitor 102 may receive electrical signals corresponding to specific values of the patient 117 that may be utilized in calculation of specific physiological parameters of the patient 117 simultaneously. That is, the detectors 118A may comprise a four-channel detector array that allows for determination of the oxygen saturation of a patient, the hematocrit levels of a patient, the blood/glucose levels of a patient, and/or other physiological readings of the patient, simultaneously. Accordingly, each channel line may transmit electrical signals corresponding to each of the above-referenced values for calculation by the monitor 102. Additionally, more or fewer detectors than illustrated detectors 118A-D may be utilized as part of the detector array to receive the light from the broadband emitter 146 and to transmit the electrical signals corresponding to the light in specific wavelengths to the monitor 102 for calculation of variety of physiological parameters.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may cover all modifications, equivalents, and alternatives falling within the spirit and scope of the disclosure as defined by the following appended claims.

What is claimed is:

1. A physiological sensor comprising:
a broadband light emitter adapted to transmit light across a range of wavelengths;
an optical filter associated with the broadband emitter, wherein the optical filter is adapted to substantially pass light transmitted from the broadband light emitter at a specific wavelength or at a subset of the range of wavelengths through the optical filter and to substantially block light at all other wavelengths from passing through the optical filter; and
a light detector adapted to receive the light passed through the optical filter.

2. The physiological sensor, as set forth in claim 1, comprising:
a second broadband light emitter adapted to transmit light across a range of wavelengths; and
a second optical filter associated with the second broadband emitter, wherein the second optical filter is adapted to substantially pass light transmitted from the broadband light emitter at a second specific wavelength or at a second subset of the range of wavelengths through the second optical filter and to substantially block light at all wavelengths from passing through the second optical filter.

3. The physiological sensor, as set forth in claim 2, wherein the specific wavelength or subset is in a red range suitable for pulse oximetry measurements.

4. The physiological sensor, as set forth in claim 2, wherein the second wavelength or second subset is in an infrared range suitable for pulse oximetry measurements.

5. The physiological sensor, as set forth in claim 1, wherein the optical filter comprises filter glass, deposited on to the broadband emitter.

6. The physiological sensor, as set forth in claim 1, wherein the optical filter is disposed adjacent the broadband emitter and wherein the optical filter and the broadband emitter comprise separate discrete components.

7. A pulse oximetry system comprising:
a pulse oximetry monitor; and
a sensor assembly configured to be coupled to the monitor, the sensor assembly comprising:
a broadband light emitter adapted to transmit light across a range of wavelengths;
a plurality of light detectors adapted to receive the light from the broadband emitter; and
a plurality of optical filters, wherein each of the plurality of optical filters is associated with a single one of the plurality of light detectors, and wherein each of the plurality of optical filters is adapted to substantially pass light transmitted from the broadband light emitter at a specific wavelength or at a subset of the range of wavelengths to the associated single one of the plurality of light detectors and to substantially block light at all other wavelengths from the associated single one of the plurality of light detectors.

8. The pulse oximetry system, as set forth in claim 7, comprising a first channel line configured to couple a first light detector of the plurality of light detectors to the monitor.

9. The pulse oximetry system, as set forth in claim 7, comprising a second channel line configured to couple a second light detector of the plurality of light detectors to the monitor, wherein the second channel line is independent from the first channel line.

10. The pulse oximetry system, as set forth in claim 7, wherein the specific wavelength or subset of the range of wavelengths differs for each of the plurality of optical filters.

11. The pulse oximetry system, as set forth in claim 7, wherein the specific wavelength or subset is in a red range suitable for pulse oximetry measurements.

12. The pulse oximetry system, as set forth in claim 7, wherein the second wavelength or second subset is in an infrared range suitable for pulse oximetry measurements.

13. A method comprising:
transmitting light with a plurality of wavelengths via a broadband light emitter;
filtering the transmitted light via an optical filter adapted to pass light at a specific wavelength or at a subset of the range of wavelengths and to substantially block light at all other wavelengths from passing through the optical filter to generate filtered light;
receiving the filtered light at a light detector; and
calculating physiological parameters based on the filtered light.

14. The method of claim 13, comprising displaying indications of the physiological parameters on a pulse oximeter.

15. The method of claim 13, wherein the filtering is performed at the broadband emitter.

16. The method of claim 13, wherein the filtering is performed at the light detector.

17. The method of claim 13, comprising:
filtering the transmitted light via a second optical filter adapted to pass light at a second specific wavelength or at a second subset of the range of wavelengths and to substantially block light at all other wavelengths from passing through the second optical filter to generate second filtered light; and
receiving the second filtered light at a second light detector.

18. The method of claim 17, comprising:
generating first reception signals at the light detector based on the filtered light;
generating second reception signals at the second light detector based on the second filtered light; and
transmitting the first reception signals and the second reception signals to a monitor on independent channel lines.
